(19) United States
(12) Patent Application Publication
(10) Pub. No.: US 2002/0165548 A1
(43) Pub. Date: Nov. 7, 2002

(54) Medical device

(75) Inventor: Rajwinder S. Jutley, Mapperley (GB)

Correspondence Address: JENKENS & GILCHRIST, A PROFESSIONAL CORPORATION, 1100 LOUISIANA, SUITE 1800, HOUSTON, TX, 77002-5214, US

(73) Assignee: Grampian University Hospitals NHS Trust,, Aberdeen (GB)

(21) Appl. No.: 10/047,594

(22) Filed: Jan. 14, 2002

Related U.S. Application Data
(63) Continuation of application No. PCT/GB00/02699, filed on Jul. 14, 2000.

(30) Foreign Application Priority Data
Jul. 19, 1999 (GB) GB 9916724.9
May 31, 2000 (GB) GB 0013140.0

(51) Int. Cl. A61B 17/58

(52) U.S. Cl. 606/73

(57) ABSTRACT

A body-implantable device is disclosed for use in closing sternotomy openings, the device comprising a shaft with a conduit therethrough to allow the passage of a suture through the shaft, and a bone-gripping formation such as a thread or array of ridges or wedges on a surface of the shaft.

### PRIOR RELATED APPLICATIONS

This application is a continuation of previously filed foreign application No. PCT/GB00/02699, filed Jul. 14, 2000, which claims priority to previously filed foreign applications No. GB 0013140.9, filed May 31, 2000 and No. GB 9916724.9, filed Jul. 19, 1999, incorporated herein by referenced in their entirety.

### FEDERALLY SPONSORED RESEARCH STATEMENT

Not applicable.

### REFERENCE TO MICROFICHE APPENDIX

Not applicable.

### FIELD OF THE INVENTION

This invention relates to a medical device, and particularly to a device and method for closing the sternum after it has been opened for surgical procedures to be carried out e.g. on the heart.

### BACKGROUND OF THE INVENTION

Median sternotomy is used for most cardiac operations. The use of sternal wire fixation to close a median sternotomy was first described by Milton 102 years ago¹. The method, which involves the approximation of the sternal edges by twisting six or more stainless steel wires, still remains the most widely accepted technique. In conventional techniques, stainless steel wire (e.g. No 5 grade) is threaded onto a sharp curved needle which is then forced through the sternum at each side. The wires can then be pulled tight and twisted to close the sternum.

Despite its widespread use, sternal wire fixation is not without its morbidity and mortality. Serious complications include sternal dehiscence (i.e. spontaneous bursting open of the sternum) occurring at about 2.4% incidence and mediastinitis at 0.25%^{2,3}. In addition, sternal malunion and nonunion contributing to excessive sternotomy site movement worsens postoperative pain leading to decreased inspiratory effort. This predisposes to postoperative atelectasis and chest infections with inherent complications.

With recent rapid advances in cardiac techniques, an increasing number of patients with coexisting disease are being offered surgery. The older population with osteoporosis and patients with chronic obstructive pulmonary disease are now routinely being operated on. This increase in the range of patients for cardiac surgery also suggests a greater proportion of candidates on steroids and with diabetes, both recognised risk factors for impaired wound and bone healing.

The prevention of sternal dehiscence and sternal infection following a sternotomy remains a challenge to the cardiac surgeon. Several authors have investigated alternative and more rigid methods of sternal fixation following median sternotomy. These mainly involve reinforced sternal struts attached to the sternum; more complex wiring patterns where the wires are laced over each other in order to reduce the strain on any one wire; flat sutures of plastic instead of wire that present a larger surface area against the sternum and therefore exert less pressure on it per suture; and polyester and steel, and metal plate^{2, 4-8}.

### SUMMARY OF THE INVENTION

Embodiments of the invention provide a device for use in joining a portion of bone material to another object. The device comprises a shaft for extending through the portion of bone material, a conduit through the shaft for passage of a closure device therethrough, and a formation on a surface of the shaft that is adapted to grip the bone material. There is also provided a method of joining a portion of bone material to another object. The method comprises (a) providing a device having a shaft, a conduit through the shaft and a bone engaging formation on a surface of the shaft; (b) placing the device in the portion of bone material to be joined so that it is retained therein by means of the bone engaging formation; (c) passing a closure device through the conduit in the device so as to pass through the portion of bone material; (d) attaching the closure device to the other object; and (e) making up the closure device to join the portion of bone material to the other object.

Additional aspects and details of the embodiments of the invention, as well as advantages thereof, are described in the following or become apparent to those skilled in the art with the following description.

### BRIEF DESCRIPTION OF THE DRAWINGS

FIG. 1a is a schematic sectional view through a first device.

FIG. 1b is a schematic sectional view through a second device.

FIG. 2 is a perspective view of a third device.

FIG. 3 is a general schematic sectional view through a sternum showing the FIG. 2 device in use.

FIG. 4 is a schematic plan view of the FIG. 2 device showing the principles of Hertzian contact analysis.

FIG. 5 is a graph showing variation of mean pressure between wire and sternum with diameter of wire for chest pressure of 50, 100 and 300 mmHg.

FIG. 6 shows views from one side and beneath the FIG. 2 device.

FIG. 7 shows a schematic drawing of a model used comparative testing of the FIG. 2 device.

FIG. 8 shows a further schematic drawing of a model used for testing the FIG. 2 device.

FIG. 9 shows boxplots of the failure loads using wire alone and wire plus a FIG. 2 device.

### DESCRIPTION OF EMBODIMENTS OF THE INVENTION

According to the present invention there is provided a device for use in joining a portion of bone material to another object, comprising a shaft for extending through the portion of bone material, a conduit through the shaft for passage of a closure device therethrough, and a formation on a surface of the shaft that is adapted to grip the bone material.

The invention also provides a method of joining a portions of bone material to another object, the method comprising:

providing a device having a shaft, a conduit through the shaft and a bone engaging formation on a surface of the shaft;

placing the device in the portion of bone material to be joined so that it is retained therein by means of the bone engaging formation;

threading a closure device through the conduit in the device so as to pass through the portion of bone material;

attaching the closure device to the other object; and

making up the closure device to join the portion of bone material to the other object.

Typically the method is used to join two portions of bone material together, so that the other object is typically another portion of bone material. Typically a respective device is provided in each portion of bone material to be joined.

Typically the two portions of bone material comprise two portions of sternum, as the preferred embodiment of the invention comprises a device for closing a sternotomy after cardiac surgery.

In certain embodiments the bone-engaging and gripping formation can simply be one or more ridges on the surface of the shaft that can be arranged parallel to one another or helically like a screw thread. Preferred embodiments of the invention have a simple screw thread on their outer surface because they can thereby be made self tapping and therefore can simply be driven into the bone etc by means of a screwdriver. However, other embodiments can be placed in pre-drilled holes in which they can be retained by the simple ridges etc or other bone-engaging formations.

The closure device can be a conventional suture such as a stainless steel wire length that can be twisted to close it about the wound etc. The closure device can be mounted on an insertion device e.g. threaded onto a needle or can simply be passed through the conduit without being mounted on any insertion device. Good results can be obtained simply by using tapes and bands or plastics or other materials. Sternaband closures are also suitable.

One embodiment of the invention comprises a simple cannulated screw that can be placed on either side of the sternotomy. Conventional stainless steel wire can be passed through the cannula of each screw and the sternotomy can be closed in the usual manner.

Embodiments of the invention have the advantage that closing a median sternotomy with cannulated screws plus wire should reduce the occurrence of sternal dehiscence.

Preferably the shaft has a screw thread (or other formations) on its outer surface. However certain embodiments may comprise an annular shaft with an internal screw thread. A typical thread can be manufactured according to BS ISO 5835, which is incorporated herein by reference. Flutes can optionally be cut into the threads to make it self tapping.

In certain embodiments of the invention the conduit through the shaft is a straight bore. In certain embodiments the bore can have chamfered edges to reduce strain on the wire or other closure device passing through the conduit.

The device can be formed from stainless steel or from plastics materials. Inplantable grade stainless steel is a useful material and can be employed as described in BS 7252-1 (1997), which is incorporated herein by reference.

Typical embodiments of devices should comply with BS EN ISO 14602, which is incorporated herein by reference. Risk analysis can typically be carried out in accordance with BS EN 1441 (1998), which is incorporated herein by reference. Typically the device has a length to suit the bone portion into which it will be located, and should not extend beyond the portion, so that it is flush with, or contained wholly within, the bone portion in which it is embedded so as to prevent damage to organs. It should typically be manufactured from the same material as the wire with which it will be used e.g. stainless steel 316L), or at least from a material that will not react chemically with the wire. In preferred embodiments of the invention, sharp edges of the screw can typically be removed so that the wire is not damaged when force is applied to it in use.

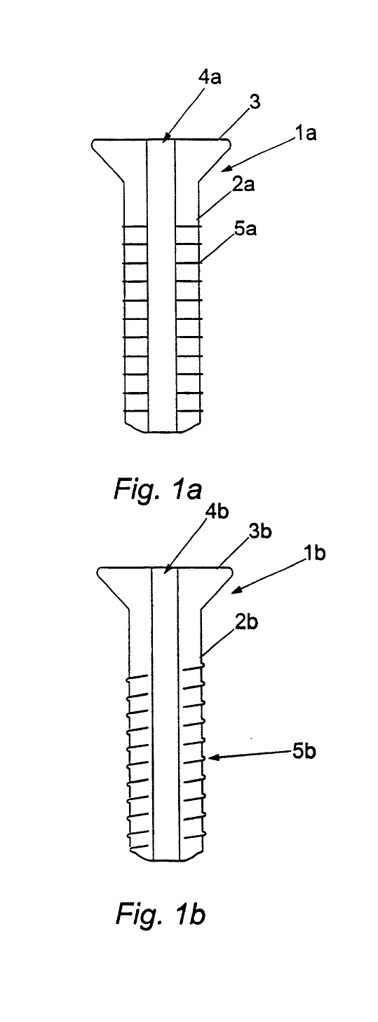

An embodiment of the invention will now be described by way of example and with reference to the accompanying drawings 1-9. Referring now to the drawings, a screw 1 for use in joining a portion of bone material to another object has a shaft 2, a head 3, a conduit 4 passing through the head 3 and shaft 2, and a formation 5 located on the outer surface of the shaft 2. In the FIG. 1a embodiment the formation comprises an array of mutually parallel annular ridges 5a extending around the shaft 2, whereas in the FIG. 1b embodiment the formation comprises a continuous helical screw thread 5b extending around the shaft 2. The sectional view shows the conduit in the centre of the shaft 2, and does not indicate that the thread 5b or ridges 5a are interrupted, but this remains an option for these embodiments. Note that instead of annular ridges or helical threads, the formations 5 could comprise wedges pointing downwards along the shaft 2 with the narrow ends of the wedges pointing away from the head 3.

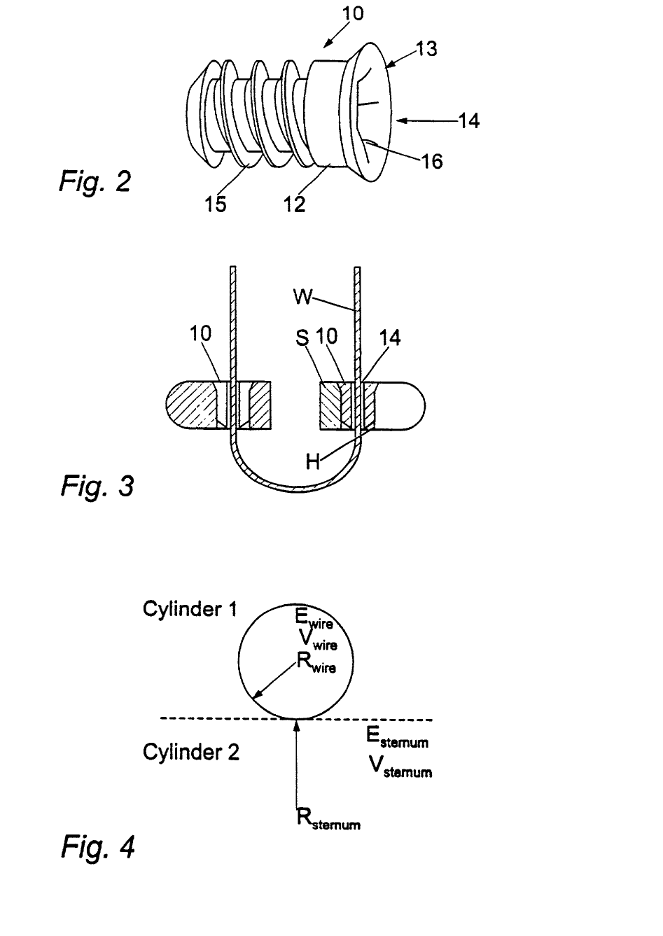

FIG. 2 shows a second embodiment of a screw 10 having a shaft 12, a head 13, a conduit 14 through the head 13 and shaft 12, and a helical thread 15 extending around the shaft 12. The helical thread is optionally self tapping, and has a typical outer diameter of around 4-8 mm, and typically 6 mm. The head 13 has a profiled socket 16 to receive a screwdriver blade or Allen key.

The screw 10 is inserted into pre-drilled holes H in a sternum S after a sternotomy, and is driven into place by a screwdriver or Allen key applied to the head 13. The screw 10 need not be placed in a pre-drilled hole H, but as it can optionally be self tapping, can simply be driven into the bone of the sternum S. Once a screw 10 has been driven flush with the sternum S on each side of the sternotomy wound, a single wire W is passed through the conduit 14 in each screw 10, and the ends of the wire are twisted together to join the wound. Conventional lacing patterns can be used in arrays of screws 10 along the sides of the wound. Conventional grade 5 stainless steel wire can be used.

The interaction between the stainless steel wire W and the sternum S was modelled as two cylinders in contact, as shown in FIG. 4. Cylinder 1 represents the wire W with a radius R_{wire}, Young's modulus E_{wire} and Poisson's ratio ν_{wire}. Cylinder 2 represents the sternum S with a radius Rₛₜₑᵣₙᵤₘ, Young's modulus Eₛₜₑᵣₙᵤₘ and Poisson's ratio νₛₜₑᵣₙᵤₘ. If the wire W is pressed in contact with the sternum S, as would occur in a sternotomy closure, by a force F per unit length, the problem is two-dimensional. The mean pressure between the wire W and the sternum S can be determined from Hertzian contact analysis [Johnson, 1985] by the equation:

$${P}_{mean} = \frac{π}{4} {( \frac{F {E}^{*}}{π R} )}^{1 / 2} \qquad Equation ( 1 )$$

where F=force per unit length

$${E}^{*} = combined elastic constant where \frac{1}{{E}^{*}} = \frac{1 - {v}_{wire}^{2}}{{E}_{wire}} + \frac{1 - {v}_{sternum}^{2}}{{E}_{sternum}} \qquad Equation ( 2 )$$

R=relative radius of curvature where

$$\frac{1}{R} = \frac{1}{{R}_{wire}} + \frac{1}{{R}_{sternum}} \qquad Equation ( 3 )$$

In order to calculate the mean pressure acting between the wire W and the sternum S it was necessary to determine the unknown variables in equation 1, namely F, E* and R. The values used are described in the next sections.

Combined Elastic Constant (E*)

The material properties of bone can vary considerably and no absolute values can be quoted [Reilly & Burstein; Rho et al; Zioupos & Currey; Zysset et al]. Indeed, Zioupos & Currey [1998] have show that the Young's modulus of cortical bone decreases with age. However, for this analysis some average values determined by Reilly and Burstein [1975] for human femoral bone were used: a Young's modulus (Eₛₜₑᵣₙᵤₘ) of 17 GPa and a Poisson's ratio (νₛₜₑᵣₙᵤₘ) of 0.46. The stainless steel wire had a Young's modulus (E_{wire}) of 200 GPa and a Poisson's ratio (ν_{wire}) of 0.29 [Gere and Timoshenko, 1985]. Substituting the material properties for bone and steel wire into equation (2) we get:

$$\frac{1}{{E}^{*}} = \frac{1 - {v}_{wire}^{2}}{{E}_{wire}} + \frac{1 - {v}_{sternum}^{2}}{{E}_{sternum}} = \frac{1 - {0.29}^{2}}{200 \times {10}^{9}} + \frac{1 - {0.46}^{2}}{17 \times {10}^{9}} ∴ {E}^{*} = 2 \times {10}^{10} N \text{/} {m}^{2}$$

Relative Radius of Curvature (R)

It can be assumed that the radius of the sternum is infinite and therefore Rₛₜₑᵣₙᵤₘ=∞. Substituting into equation (3) we get:

$$\frac{1}{R} = \frac{1}{{R}_{wire}} + \frac{1}{{R}_{sternum}} = \frac{1}{{R}_{wire}} + \frac{1}{∞} ∴ R = {R}_{wire}$$

It was decided to vary the radius of the wire to see how the pressure between the wire and sternum varied.

Force Per Unit Length (F)

The force across a sternotomy closure is given by Casha et al. [1999] as:

T=rLP Equation (4)

where T is the resultant force required to keep the sternum closed, r is the radius of the chest, L is the height of the thoracic cavity and P is the distending pressure.

Values suggested by Casha et al. [1999] for the radius of the chest (r) and the height of the thoracic cavity (L) are 0.15 m and 0.25 m, respectively. During coughing, the distending pressure can reach 300 mmHg (40 kPa) [Casha et al., 1999]. Substituting these values into equation (4) we get:

T=rLP=0.15×0.25×40000=1500 N

Thus, the total force required to keep the sternum closed is 1500 N. Since it is common to close the sternum with six wires, the force acting on each individual wire would be 250 N. For this analysis we need to know the force per unit length (F), i.e. dividing the force by the length it acts over. It can be assumed, from surgical experience, that the sternum has a thickness of approximately 0.01 m. Therefore, the force per unit length acting between the wire and the sternum will be 25 kN/m.

The value of pressure given by Casha et al. [1999] is likely for subjects with a large chest and a strong cough. Sacker [1988] suggests other values of pressure during coughing as 50 to 100 mmHg or more. Therefore, the force per unit length for these pressures would be 4.2 and 8.3 kN/m, respectively.

Calculations

The above values of the combined elastic constant (E*), relative radius of curvature (R_{wire}) and force per unit length (F) were substituted into equation (1) to give the mean pressure between the wire and the sternum at pressures of 50, 100 and 300 mmHg. The radius of the wire (R_{wire}) was varied between 0.05 and 5 mm to see how the pressure varied. It should be noted that stress is equal to the local pressure [Johnson, 1985], hence, pressure and stress are interchangeable terms in the context of this disclosure.

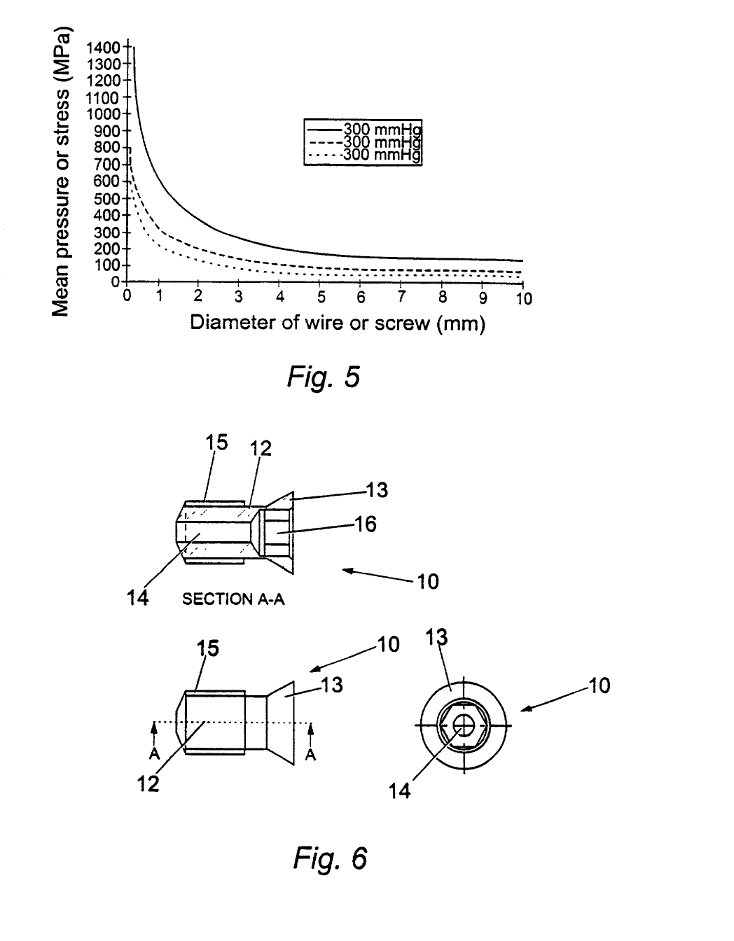

FIG. 5 shows a graph of mean pressure (or stress) between the wire and the sternum against diameter of wire at distending pressures of 50, 100 and 300 mmHg. It can be seen that if the diameter of wire is small the mean pressure is high. With increasing diameter of the wire the pressure decreases.

The most common and widely accepted method of sternum closure is to use No. 5 stainless steel wire (Ethicon Ltd, Edinburgh, UK) which has a diameter of 0.7 mm. Using this wire and with a high distending pressure in the chest, the mean stress between the wire and the sternum is high and at a magnitude comparable with the failure stress of bone. If a patient develops a cough with a distending pressure of 300 mmHg the stress between the wire (diameter 0.7 mm) and the sternum will be 529 MPa. Reilly and Burstein [1975] report the mean ultimate compressive stress of human femur in the transverse and longitudinal directions to be 131 and 205 MPa, respectively. Zioupos and Currey [1998] have shown that failure stress of cortical bone decreases from 170 MPa, for a specimen aged 35 years, by 3.7% per decade.

The model, therefore, shows that sternal dehiscence can occur during normal physiological loading of the chest, i.e. during coughing. This was also found in a cadaveric study when distracting forces were applied across a sternotomy that had been closed using wire [McGregor et al, 1999]. Significant amounts of sternal motion were detected with the application of a physiological force.

FIG. 5 shows the potential benefit of placing a cannulated screw into the sternum rather than using conventional wire on its own. The axis for wire diameter can now be read as outside thread diameter of a cannulated screw. For example, the mean stress between the wire (diameter 0.7 mm) and the sternum with a distending pressure of 100 mmHg would be 305 MPa. If the wire was replaced with a cannulated screw (outside diameter 6 mm) and wire combination, the mean pressure between the screw and the sternum would be 104 Mpa. Using a 5 mm diameter screw the pressure would be 114 Mpa. Using a cannulated screw would reduce the contact stresses to below the fracture stress of bone and can reduce the incidence of sternal dehiscence.

FIG. 6 shows detailed views of the FIG. 2 screw 10. Screw 10 has an outer thread diameter of 6 mm, which surgical experience suggests could be fitted into the human sternum. The conduit 14 down the centre of the screw 10 has a diameter of 2 mm to allow for a 0.7 mm No. 5 stainless steel wire (Ethicon Ltd, Edinburgh) to easily pass through. It may be preferred that a range of screw diameters would be available to the surgeon e.g. 4-10 mm.

Mechanical testing was undertaken to compare using wire W on its own with a screw 10 plus wire W. Blocks B of balsa wood 120×15×12 mm were used as a sternum substitute. For testing the wire W on its own, a hole of diameter 1.4 mm was drilled into the centre of the block B. This diameter is the size of the main part of a conventional needle (Ethicon Ltd, Edinburgh) that is currently used to insert the wire using conventional methods. For testing the sternum screw, a hole of diameter 5 mm was drilled into the centre of the block B. A screw 10 could then be driven in the drilled hole. Clamps were used to secure the wood to a plate, mounted on the base of an Instron materials testing machine (Instron Ltd, High Wycombe, UK).

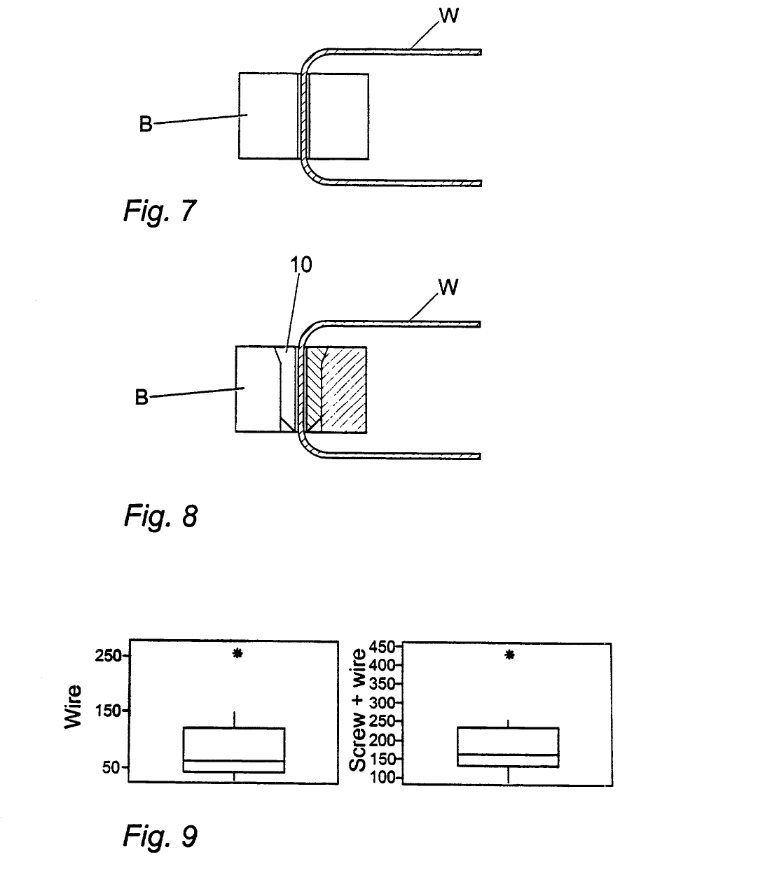

A loop of No 5 stainless steel wire (Ethicon Ltd, Edinburgh, UK) was passed through the hole in the wood, as shown in FIG. 7, and around a bar mounted on the actuator of the testing machine. The end of the wire W was then twisted to close the loop. The actuator of the testing machine was set to rise at a rate of 25 mm/min. As the actuator rose, a tension was applied to the wire. Each test continued until the wire cut through the wood. The force at failure was noted. Ten tests were undertaken using wire on its own.

The whole procedure was then repeated for the block B fitted with the screw 10 as shown in FIG. 8. This time the wire was passed through the conduit 14 in the screw 10. Ten tests were carried out using wood fitted with the sternum screw, as shown in FIG. 8. The force at failure, as the screw 10 cut through the wood, was noted.

When the wire W was placed through the balsa wood on its own, the tension applied to the wire W resulted in the wire cutting through the wood at a mean load of 103.9 N. When the tests were undertaken with the screw 10 fitted to the balsa wood, the screw 10 cut through the wood at a mean load of 208.7 N. The descriptive statistics for the tests are shown in Table 1. The data from the two tests were not normally distributed as assessed using the Anderson-Darling normality test. A Mann-Whitney test was, therefore, used as a significance test. It was found that there was a significant (P=0.007) difference between the median values of the two tests. Thus the screw-plus-wire combination was stronger than wire on its own.

Table 1 Descriptive statistics for the failure load for tests undertaken with wire and screw plus wire.

TABLE 1

| | n | Mean (N) | SD (N) | Median | Min (N) | Max (N) |
|---|---|---|---|---|---|---|
| Wire | 10 | 103.9 | 73.5 | 77.4 | 38.8 | 278.9 |
| Screw + wire | 10 | 208.7 | 92.6 | 188.1 | 107.6 | 439.4 |

On closer examination of the data, as shown in the boxplots in FIG. 9, it was found that there was an outlier (greater than two standard deviations from the mean) in each set of data. The outliers were removed to investigate if they would influence the results. The mean failure load using wire on its own and for using the screw 10 plus wire reduced to 84.4 N and 183.1 N, respectively. The descriptive statistics are shown in Table 1. Removing the outliers meant that the data were now normally distributed. A two-sided two-sample t-test showed a significant (P=0.0003) difference between the mean values. Therefore, these outliers did not influence the results.

Table 2 Descriptive statistics for the failure load for tests undertaken with wire and screw plus wire with the outliers removed.

TABLE 2

| | n | Mean (N) | SD (N) | Median | Min (N) | Max (N) |
|---|---|---|---|---|---|---|
| Wire | 9 | 84.4 | 42.7 | 62.6 | 38.8 | 165.6 |
| Screw + wire | 9 | 183.1 | 47.6 | 185.9 | 107.6 | 250.8 |

The comparative data indicates that where wire is used alone to close a sternotomy, the stress between the wire and the sternum can be comparable to the breaking strength of bone. The model shows that using a screw-plus-wire will mean that the stress between the screw and the sternum can satisfactorily be below the breaking strength of bone.

Examination of the mean failure load showed that using a sternum screw is roughly twice as strong as using wire on its own.

Certain embodiments of the invention have advantages over conventional systems. For example, post operative bleeding following a sternotomy is frequently a problem requiring reopening of the sternum to reinvestigate the chest for the cause of the bleeding. Re-closing the sternum is easier with certain embodiments of the invention that allow the insertion of the wires back into the same holes i.e. the conduits through the screws that can remain in place in the sternum. This obviates the need to perforate the sternum at other places, thereby weakening it further. This is also a benefit for “redo” surgery that has become more commonplace in recent years in that patients requiring cardiac surgery early in life frequently need a further similar operation after about 15-20 years. The same devices implanted in these patients can be used to close the patients in the “redo” operation.

A further advantage is that certain screw-threaded embodiments can be removed from the sternum easily and without significant damage to the sternum during the procedure. This is very useful when patients who have received the implants develop nickel allergy following the procedure.

### References

1. Milton H. Mediastinal surgery. Lancet 1897;1:872-5

2. Sirivella S, Zikria E A, Ford W B, Samadani S R, Miller W H, Sullivan M E. Improved technique for closure of median sternotomy incision: Mersilene tapes versus standard wire closure. J Thorac Cardiovasc Surg 1987;94:591-5

3. Baskett R J, MacDougall C E, Ross D B. Is mediastinitis a preventable complication? A 10-year review. Ann Thorac Surg 1999;67:462-5

4. Scovotti C A, Ponzone C A, Leyro-Diaz R M. Reinforced sternal closure. Ann Thorac Surg 1991;51:844-5

5. Ozaki W, Buchman S R, Iannettoni M D, Frankenburg E P. Biomechanical study of sternal closure using rigid fixation techniques in human cadavers. Ann Thorac Surg 1998;65:1660-5

6. Soroff H S, Hartman A R, Pak E, Sasvary D H, Pollak S B. Improved sternal closure using steel bands: early experience with three-year follow-up. Ann Thorac Surg 1996;61:1172-6

7. Taber R E, Madaras J. Prevention of sternotomy wound disruptions by use of figure-of-eight pericostal sutures. Ann Thorac Surg 1969;8:367-9

8. LeVeen H L, Piccone V A. Nylon-band closure. Arch Surg 1968;96:36-9

9. Johnson K L. Contact mechanics. Cambridge: Cambridge University Press, 1985.

10. Reilly D T, Burstein A H. The elastic and ultimate properties of compact bone tissue. J Biomech 1975;8:393-405.

11. Rho J-Y, Tsui T Y, Pharr G M. Elastic properties of human cortical and trabecular lamellar bone measured by nanoindentation. Biomaterials 1997;18:1325-30.

12. Zioupos P, Currey J D. Changes in the stiffness, strength and toughness of human cortical bone with age. Bone 1998;22:57-66.

13. Zysset P K, Guo X E, Hoffler E, Moore K E, Goldstein S A. Elastic modulus and hardness of cortical and trabecular bone lamellae measured by nanoindentation in the human femur. J Biomech 1999;32:1005-12.

14. Gere, J M, Timoshenko S P. Mechanics of Materials, Second SI edition. Massachusetts: VNR International, 1987.

15. Cassia A R, Yang L, Kay p H, Sale M, Cooper G J. A biomechanical study of median sternotomy closure techniques. Eur J Cardiothorac Surg 1999;15:365-9.

16. Sackner M A. Cough. In: J F Murray & J A Nadel, eds. Textbook of respiratory medicine. Philadelphia: W B Saunders, 1988;397-408.

17. McGregor W E, Trumble D R, Magovern J A. Mechanical analysis of midline sternotomy wound closure. J Thorac Cardiovasc Surg 1999;117:1144-50.

18. BS 7252-1 (1997). Metallic materials for surgical implants. Specification for wrought stainless steel. British Standards Institute, London.

19. BS EN 1441 (1998). Medical Devices—Risk Analysis. British Standards Institute, London.

20. BS EN ISO 14602 (1998). Non-active surgical implants. Implants for osteosynthesis. Particular requirements. British Standards Institute, London.

21. BS ISO 5835 (1997). Implants for osteosynthesis. Bone screws and auxiliary equipment. Specification for the dimensions of screws having hexagonal drive connection, spherical under-surfaces and asymmetrical thread. British Stands Institute, London.

What is claimed is:

1. A device for use in joining a portion of bone material to another object, the device comprising a shaft for extending through the portion of bone material, a conduit through the shaft for passage of a closure device therethrough, and a formation on a surface of the shaft that is adapted to grip the bone material.

2. A device as claimed in claim 1, wherein the other object comprises a second portion of bone material.

3. A device as claimed in claim 1, wherein the portion of bone material comprises a portion of sternum.

4. A device as claimed in claim 1, wherein the bone-gripping formation comprises at least one ridge on the surface of the shaft.

5. A device as claimed in claim 4 wherein at least two ridges are provided, arranged mutually parallel to one another.

6. A device as claimed in claim 4, wherein the ridge is annular and extends substantially around the surface of the shaft.

7. A device as claimed in claim 1, wherein the bone-gripping formation comprises a helical thread.

8. A device as claimed in claim 7, wherein the thread is self-tapping.

9. A device as claimed in claim 1, wherein the conduit through the shaft is chamfered to resist damage to the closure device.

10. A device as claimed in claim 1, made from stainless steel.

11. A device as claimed in claim 1, made from plastics material.

12. A device as claimed in claim 1, made from material capable of being absorbed in a body.

13. A device as claimed in claim 1, wherein the device comprises an annular shaft with an internal screw thread.

14. A device as claimed in claim 1, wherein the device comprises a shaft with an external screw thread.

15. A method of joining a portion of bone material to another object, the method comprising:
providing a device having a shaft, a conduit through the shaft and a bone engaging formation on a surface of the shaft;
placing the device in the portion of bone material to be joined so that it is retained therein by means of the bone engaging formation;
passing a closure device through the conduit in the device so as to pass through the portion of bone material;
attaching the closure device to the other object; and
making up the closure device to join the portion of bone material to the other object.

16. A method as claimed in claim 15, comprising the further steps of drilling a hole in the portion of bone material, and placing the device in the drilled hole.

17. A method as claimed in claim 15, wherein the device is capable of forming a hole in the portion of bone material, and is inserted therein without first making a hole.

18. A method as claimed in claim 15, wherein the other object comprises a second portion of bone material.

19. A method as claimed in claim 15, wherein the closure device and the device comprise the same material.

20. A method as claimed in claim 15, wherein one of the closure device and the device comprise a plastics material.

21. A method as claimed in claims 15, wherein the closure device and the device each comprise materials that do not react chemically with one another.

22. A method as claimed in claim 15, wherein the portion of bone material comprises one side of a sternum, and the other object comprises the other side of the sternum.

23. A method as claimed in claim 15, wherein the closure device comprises a wire.

24. A method as claimed in claim 15, wherein the device has a length to suit the bone portion in which it will be located.

25. A method as claimed in claim 15, wherein sharp edges have been removed from the device.
